(19) United States
(12) Patent Application Publication
(10) Pub. No.: US 2024/0199590 A1
(43) Pub. Date: Jun. 20, 2024

(54) SOLID FORMS OF A SGC ACTIVATOR

(71) Applicant: Boehringer Ingelheim International GmbH, Ingelheim am Rhein (DE)

(72) Inventors: Jocelyn M. ABELLA; Ashish Subhash CHITRODA, Mumbai (IN); Joe Ju GAO, Southbury, CT (US); Stephanie Cassandra KOSNIK, Ridgefield, CT (US); David A. HIRSH, Ridgefield, CT (US); Soojin KIM, Howell, NJ (US); Wenjie LI, Lawrence Township, NJ (US); Zhibin LI, Berlin, CT (US); Thomas James OFFERDAHL, Becker, MN (US); Bing-Shiou YANG, Southbury, CT (US)

(21) Appl. No.: 18/530,255

(22) Filed: Dec. 6, 2023

Related U.S. Application Data
(60) Provisional application No. 63/431,330, filed on Dec. 9, 2022.

(51) Int. Cl. C07D 405/14

(52) U.S. Cl. CPC C07D 405/14

(57) ABSTRACT

Disclosed are solid forms of an activator of soluble guanylate cyclase (sGC). The invention also relates to methods of making these solid forms, pharmaceutical compositions comprising these solid forms, and their use for medical conditions responsive to treatment with an activator of sGC.

### FIELD OF THE INVENTION

The present inventions relate to solid forms of an activator of soluble guanylate cyclase (sGC). The invention also relates to methods of making these solid forms, pharmaceutical compositions comprising these solid forms, and their use for medical conditions responsive to treatment with an activator of sGC.

### BACKGROUND OF THE INVENTION

Compound 1 is an sGC activator and has the structure shown below:

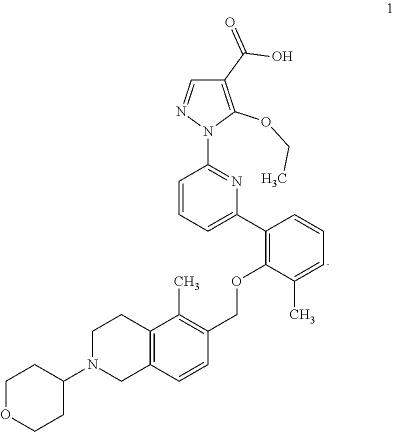 [SMILES: CCOc1c(C(=O)O)cnn1-c1cccc(-c2cccc(C)c2OCc2ccc3c(c2C)CCN(C2CCOCC2)C3)n1]

Compound 1 is useful for treating a number of kidney- and liver-related disorders including, for example, chronic kidney disease, diabetic kidney disease, nonalcoholic steatohepatitis (NASH), liver cirrhosis, and portal hypertension. Other diseases and diseases that may be treated with 1 are described, for example, in WO 2014/039434 and WO 2020/011804. The preparation of 1 is described in WO 2014/039434. (See compound 114.) However, the process described in WO 2014/039434 does not describe any particular solid form of Compound 1. Thus, there is a need for solid forms of Compound 1 having advantageous pharmaceutical properties such as, for example, processability, stability, and solubility.

### BRIEF SUMMARY OF THE INVENTION

The invention relates to novel solid forms of the Compound 1 (herein, collectively “the compounds of the invention”).

The invention also relates to methods of making the compounds of the invention and their use as activators of sGC.

In a further aspect, the present invention relates to pharmaceutical compositions, comprising a compound of the invention, optionally together with one or more inert carriers and/or diluents.

A further aspect of the present invention relates to compounds of the invention or pharmaceutical compositions comprising the compounds of the invention for the use in the prevention and/or treatment of disorders of the kidney and liver.

Yet another aspect of the present invention relates to compounds of the invention or pharmaceutical compositions comprising said compounds for use in the prevention and/or treatment of diseases or conditions which can be influenced by activation of sGC, such as chronic kidney disease, diabetic kidney disease, nonalcoholic steatohepatitis (NASH), liver cirrhosis, portal hypertension, and systemic sclerosis (scleroderma). The use comprises the manufacture of medicaments for the treatment of the corresponding diseases or disorders described herein.

In one embodiment, the invention relates to any of the crystalline forms of Compound 1 as described in Table 1 (“the compounds of the invention”).

TABLE 1
Crystalline forms of Compound 1 according to the invention.

| Solid Form | Molecular Formula |
|---|---|
| Form I | C₃₄H₃₈N₄O₅ |
| Form III | C₃₄H₃₈N₄O₅•H₂O |
| Form IV | C₃₄H₃₈N₄O₅•ACN•H₂O |
| Form V | C₃₄H₃₈N₄O₅•H₂O |

One embodiment of the invention relates to crystalline Form I of Compound 1 (“Form I”). Form I is an anhydrate and ansolvate.

In another embodiment, the invention relates to crystalline Form III of Compound 1 (“Form III”). Form III is a of 1:1 adduct with water (“monohydrate”).

In another embodiment, the invention relates to crystalline Form IV of Compound 1 (“Form IV). Form IV is a 1:1:1 adduct with acetonitrile and water.

In another embodiment, the invention relates to crystalline Form V of Compound 1 (“Form V”). Form V is a 1:1 adduct with water (monohydrate).

### BRIEF DESCRIPTION OF THE FIGURES

FIG. 1A is an X-ray powder diffraction (XRPD) pattern of Form I of Compound 1.

FIG. 1B is a ¹³C ssNMR spectrum of Form I of Compound 1.

FIG. 1C is a thermal analysis profile of Form I of Compound 1 determined by DSC measurement.

FIG. 1D is a thermal analysis of Form I of Compound 1 determined by TGA.

FIG. 1E is a Raman spectrum of Form I of Compound 1.

FIG. 2A is an XRPD pattern of Form III of Compound 1.

FIG. 2B is a ¹³C ssNMR spectrum of Form III of Compound 1.

FIG. 2C is a thermal analysis profile of Form III of Compound 1 determined by DSC measurement.

FIG. 2D is a thermal analysis profile of Form III of Compound 1 determined by TGA.

FIG. 2E is a Raman spectrum of Form IIII of Compound 1.

FIG. 3A is an XRPD pattern of Form IV of Compound 1.

FIG. 3B is a ¹³C ssNMR spectrum of Form IV of Compound 1. Asterisks denote signals from a Form I impurity.

FIG. 3C is a thermal analysis profile of Form IV of Compound 1 determined by DSC measurement.

FIG. 3D is a thermal analysis profile of Form IV of Compound 1 determined by TGA

FIG. 4A is an XRPD pattern of Form V of Compound 1.

FIG. 4B is a ¹³C ssNMR spectrum of Form V of Compound 1.

FIG. 4C is a thermal analysis profile of Form V of Compound 1 determined by DSC measurement.

FIG. 4D is a thermal analysis profile of Form V of Compound 1 determined by TGA.

FIG. 5 is an XRPD pattern of the amorphous form of Compound 1.

### DETAILED DESCRIPTION OF THE INVENTION

| Abbreviations: | |
|---|---|
| ACN | Acetonitrile |
| DSC | Differential scanning calorimetry |
| TGA | Thermal Gravometric Analysis |
| MEK | Methylethyl ketone or 2-Butanone |
| O.D. | Outside diameter |
| RH | Relative humidity |
| SSNMR | Solid-state nuclear magnetic resonance |
| THF | Tetrahydrofuan |
| XRPD | X-ray powder diffraction |

As discussed above, the invention relates to crystalline forms of Compound 1. More particularly, the invention relates to crystalline forms of Compound 1 as described in Table 1. The invention also relates to compositions comprising the crystalline compounds of the invention and the use of such compounds and compositions for treating diseases or disorders that are responsive to treatment with an activator of sGC.

In some embodiments, the invention relates to mixtures of at least two of the crystalline forms of Compound 1 as described in Table 1, compositions comprising such mixture of crystalline forms, and the use of such crystalline forms and mixtures to treat diseases or disorders that are responsive to treatment with an activator of sGC.

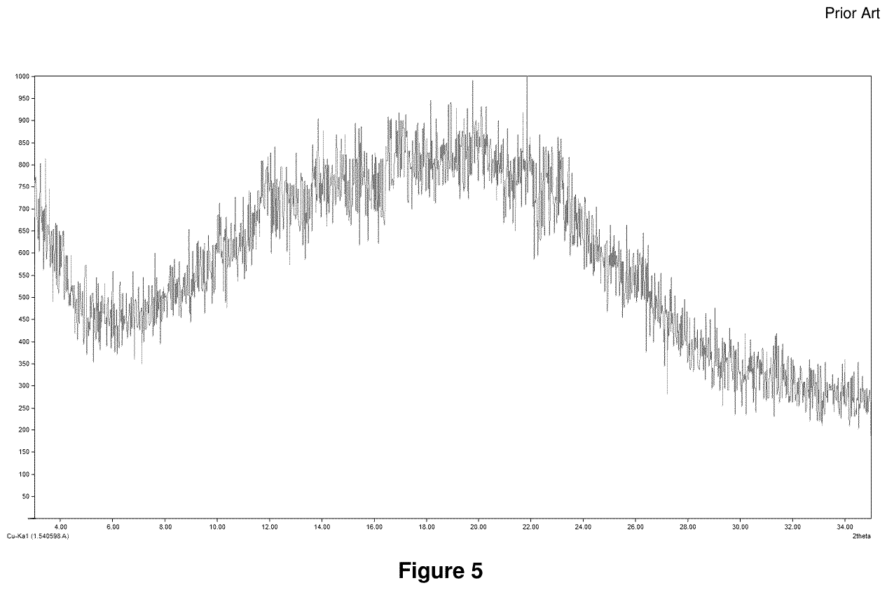

The preparation of Compound 1 is described in WO 2014/039434, but that publication makes no mention of any crystalline form of Compound 1, any method for preparing a crystalline form of Compound 1, or any of the properties of the crystalline forms of Compound 1 described herein. As described herein, Compound 1 prepared according to the method described in WO 2014/039434 is amorphous (see FIG. 5).

Form I, Form III and Form V have particularly advantageous properties (e.g., improved stability and reproducibility) as compared to the amorphous form of Compound 1. Improvements over the amorphous form include, for example, lower hygroscopicity, decreased tendency to convert to a different solid form, improved flow rates, increased bulk density, and greater resistance to mechanical attrition. For example, the amorphous form of Compound 1 has higher hygroscopicity compared to the anhydrate (Form I) and monohydrate forms (Form III, Form V), and it is not stable under high RH, in which it converting to Form Ill.

Form IV, while useful for treating the diseases or disorder described herein, is also useful as a precursor for preparing Form V.

In one embodiment, the invention relates to Form I that is substantially free of any other form of Compound 1 including the amorphous form and crystalline Forms Ill, IV and V of the invention. As used herein, “substantially free” means that the solid compound contains at least about 75% of Form I of Compound 1 based on total molar amounts of any other form of Compound 1. The amount of any form of the Compound 1 that may be present in Form I can be determined, for example, using the methods described herein.

In another embodiment, the invention relates to Form III that is substantially free of any other form of Compound 1 including the amorphous form and crystalline Forms I, IV and V of the invention. As used herein, “substantially free” means that the solid compound contains at least about 75% of Form III of Compound 1 based on total molar amounts of any other form of Compound 1. The amount of any form of the Compound 1 that may be present in Form III can be determined, for example, using the methods described herein.

In another embodiment, the invention relates to Form V that is substantially free of any other form of Compound 1 including the amorphous form and crystalline Forms I, III and IV of the invention. As used herein, “substantially free” means that the solid compound contains at least about 75% of Form V of Compound 1 based on total molar amounts of any other form of Compound 1. The amount of any form of the Compound 1 that may be present in Form V can be determined, for example, using the methods described herein.

### Characterization

The compounds of the invention can be characterized by the methods described below. Methods of preparing the compounds of the invention are described in the Experimental section.

### X-Ray Powder Diffraction (XRPD)

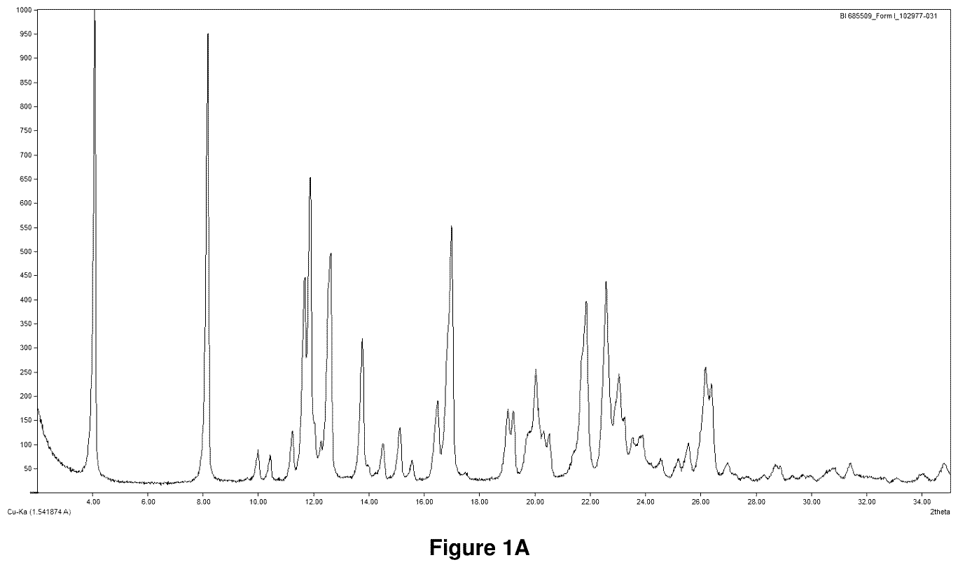

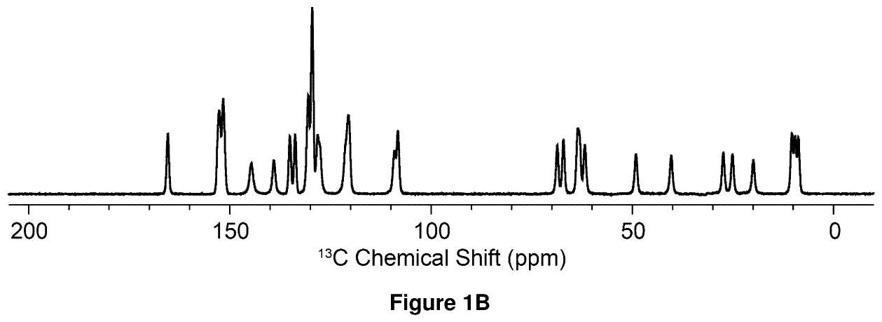

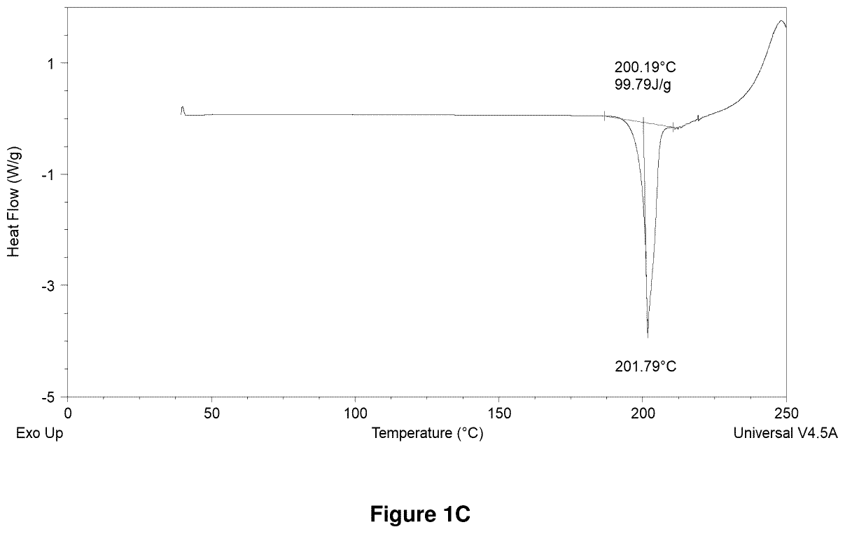

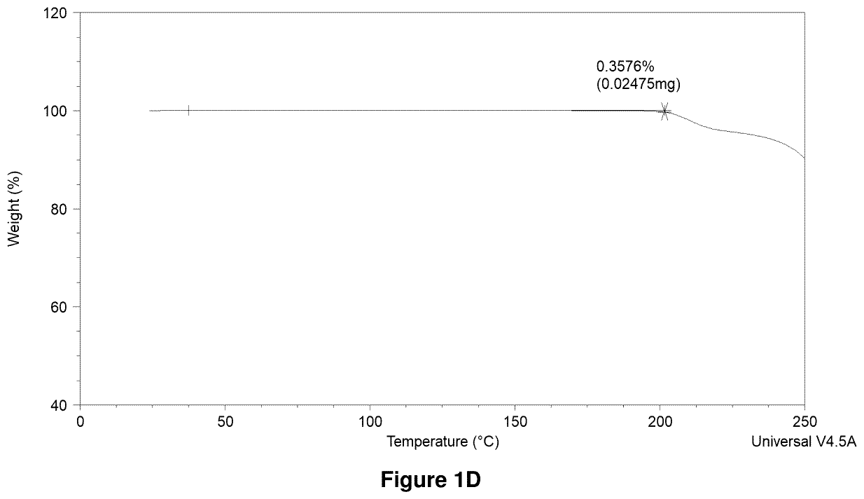

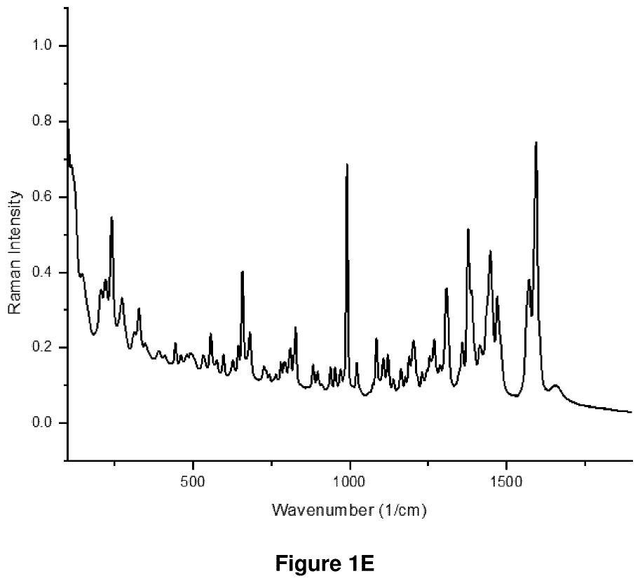

XSPR is performed with a Bruker AXS X-Ray Powder Diffractometer Model D8 Advance, using CuKa radiation (1.54A) in parafocusing mode with a graphite monochromator and a scintillation detector. Each pattern is obtained by scanning over a range of 2 degrees-35 degrees 2T, step size of 0.05 degrees 2T, step time of 4 sec per step. Exemplary XRPD spectra of the compounds of the invention are found in FIGS. 1A, 2A, 3A and 4A. An exemplary XRPD spectrum of an amorphous form of Compound 1 is shown in FIG. 5. The X-ray powder diffraction (XRPD) characteristics for the compounds of the invention reported herein have a standard deviation of ±0.2 2⊖.

### Differential Scanning Calorimetry (DSC)

DSC analysis is performed with a differential scanning calorimeter (Q2000, TA instruments, New Castle, DE), using general procedure. About 5 mg of powder was weighed into a crimped aluminum pan with pin hole. The sample is heated at 10° C./min from room temperature to 300° C. using the Q2000 DSC. Exemplary DSC traces of the compounds of the invention are found in FIGS. 1C, 2C, 3C and 4C. Results are reported below.

### Thermal Gravimetric Analysis (TGA)

TGA analysis is performed with a TA TGA 2500, TA instruments, New Castle, DE using the following general procedure. About 5 mg of powder is weighed into a platinum pan. The sample is then heated at 10□C/min from room temperature to 300□C using the TA TGA 2500.

Exemplary TGA traces of the compounds of the invention are found in FIGS. 1D, 2D, 3D and 4D. Results are reported below.

### Dynamic Vapor Sorption (DVS)

Water sorption isotherms are determined using a dynamic vapor sorption system (Advantage 1, DVS, London, UKDVS Intrinsic Plus, Surface Measurement Systems, Allentown, PA).

Approximately 5-10 mg of solid is weighed into a tared aluminum pan. The samples are subjected to 0 to 90% RH stepwise with a step size of 510% at 25° C. Equilibration criteria are dm/dt of 0.002% over 5 minutes or 360 minutes at a specified % RH. Each sample is equilibrated at each RH step for at least 60 min, and equilibrium is assumed if weight increase is less than 0.1% within one minute, and the maximum duration on each RH is 6 hours. Therefore, each sample is held at a given RH for 1 to 6 hours depending on how fast the equilibrium is reached

### ¹³C Solid-State NMR (SSNMR)

¹³C Solid-state NMR (SSNMR) data for samples of Form I, Form III (monohydrate), Form IV (ACN/H₂O solvate), and Form V (monohydrate) are acquired on a Bruker Avance III HD NMR spectrometer (Bruker Biospin, Inc., Billerica, MA) at 11.7 T (¹H=500.28 MHz, ¹³C=125.81 MHz). Samples are packed in 4 mm O.D. zirconia rotors with Kel-F(R) drive tips. A Bruker model BL4 VTN probe is used for data acquisition and sample spinning about the magic-angle (54.74 degrees). Sample spectrum acquisition uses a spinning rate of 12 kHz. A standard cross-polarization pulse sequence is used with a ramped Hartman-Hahn match pulse on the proton channel at ambient temperature and pressure. The pulse sequence uses a 4 millisecond contact pulse and a 20, 3, 10, 3.64 second recycle delay for Form I, Form III (hydrate), Form IV (ACN/H₂O solvate), and Form V (hydrate), respectively. SPINAL64 decoupling and TOSS sideband suppression are also employed in the pulse sequence. No exponential line broadening is used prior to Fourier transformation of the free induction decay. Chemical shifts are referenced using the secondary standard of adamantane, with the high frequency resonance being set to 38.48 ppm. The magic-angle is set using the ⁷⁹Br signal from KBr powder at a spinning rate of 5 kHz. Exemplary ¹³C SSNMR spectra of the samples are found in FIGS. 1B, 2B, 3B and 3C and in Tables below. The report chemical shifts have an uncertainty of ±0.3 ppm.

### Raman

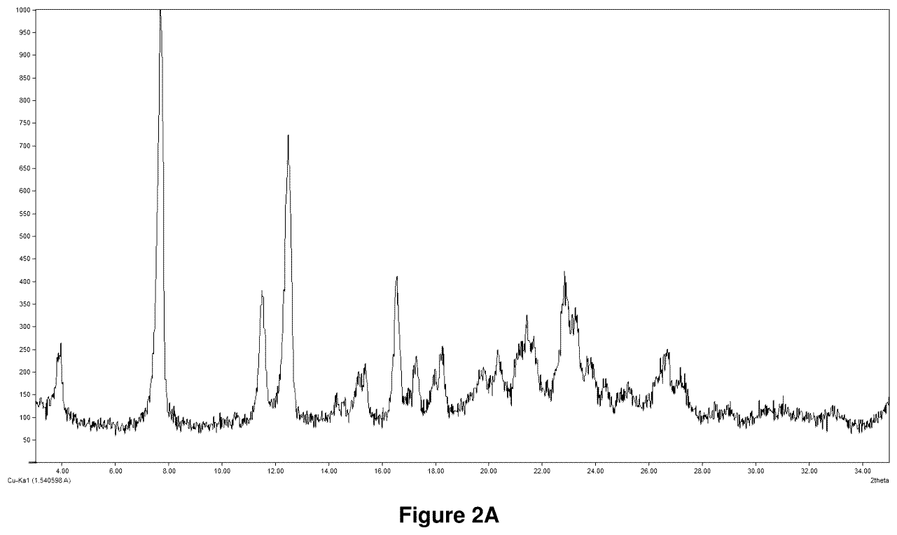

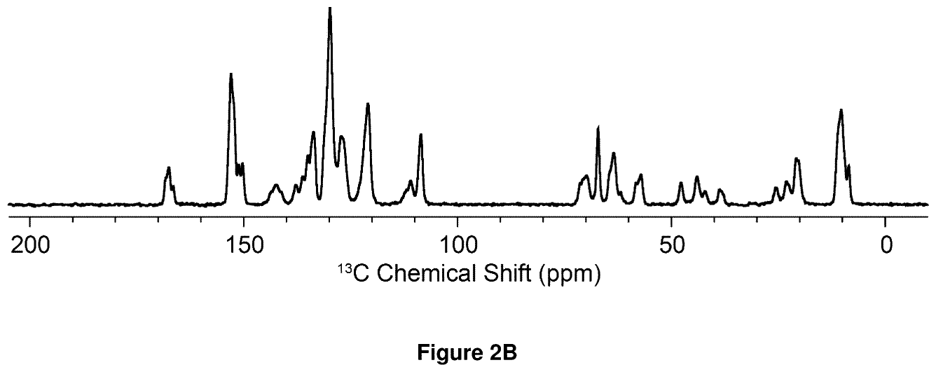

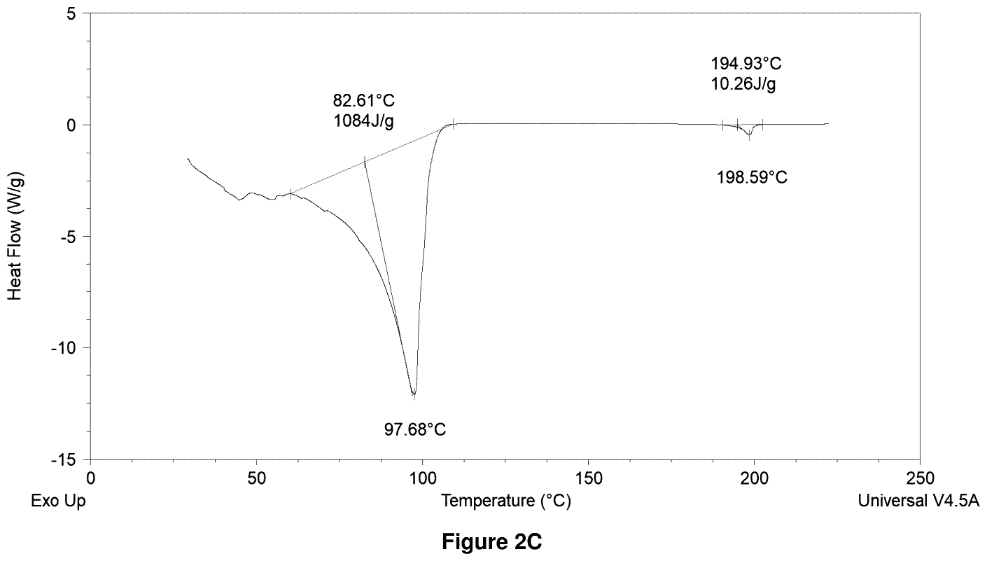

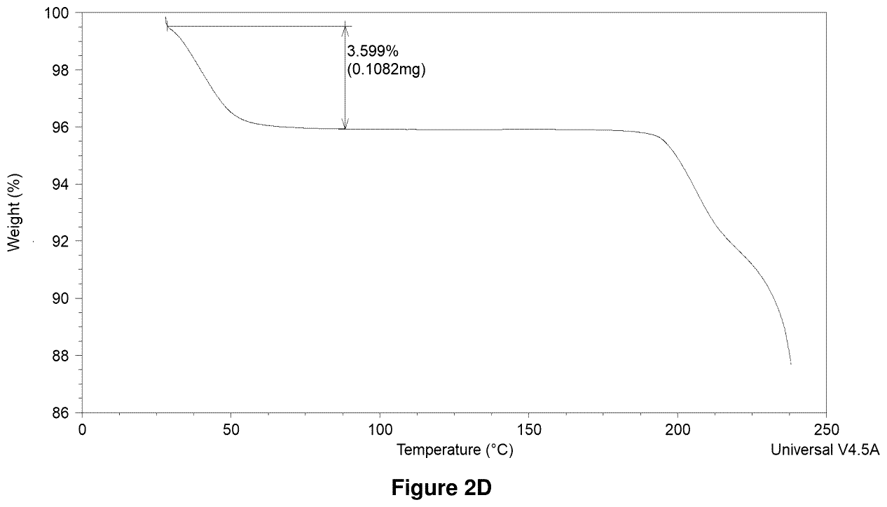

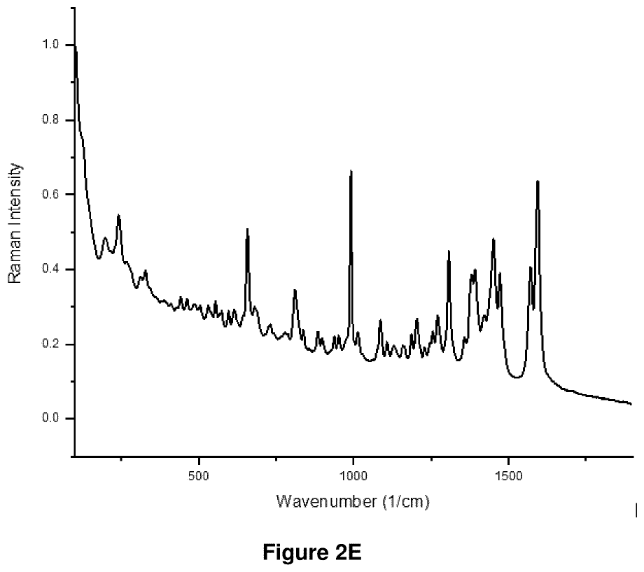

Raman data is acquired on a Transmission Raman, Agilent TRS100, SN 6032, using an 830 nm laser at 0.6 Watt power. The exposure time for the experiments is 0.4 seconds with 20 accumulations. Exemplary Raman spectra for samples of Form I and Form III of the invention are found in the FIGS. 1E and 2E and the results reported in the Tables below.

### Characteristics of Form I

The X-ray powder diffraction (XRPD) pattern of Form I of Compound 1 is shown in FIG. 1A; the ¹³C solid state NMR spectrum of Form I of Compound 1 is shown in FIG. 1B; the thermal analysis profiles of Form I of Compound 1 determined by DS, and TGA measurement are shown in FIGS. 1C and 1D, respectively; and the Raman spectrum of Form I of Compound 1 is shown in FIG. 1E.

Characteristic XRPD peaks, ¹³C solid-state nuclear magnetic resonance peaks, and Raman peaks are provided in Table 2, Table 3 and Table 4, respectively.

TABLE 2
X-ray powder diffraction (XRPD) characteristics
from FIG. 1A for Form I.

| | Intensity I/I, | | Intensity I/I, | | Intensity I/I, |
|---|---|---|---|---|---|
| 2Θ, [°] | [%] | 2Θ, [°] | [%] | 2Θ, [°] | [%] |
| 4.1 | 98.3 | 15.6 | 4.4 | 23.5 | 7.5 |
| 8.2 | 100 | 16.5 | 14.4 | 23.8 | 5.1 |
| 10.0 | 6.5 | 17.0 | 52.1 | 23.9 | 4.6 |
| 10.4 | 5.5 | 19.0 | 14.4 | 24.6 | 3.7 |
| 11.2 | 10.9 | 19.2 | 13.6 | 25.2 | 3.8 |
| 11.7 | 44.7 | 20.0 | 21.0 | 25.6 | 7.4 |
| 11.9 | 64.2 | 20.3 | 6.3 | 26.2 | 25.4 |
| 12.6 | 46.2 | 20.5 | 9.2 | 26.4 | 13.6 |
| 13.8 | 31.2 | 21.8 | 35.6 | 27.0 | 3.0 |
| 14.5 | 7.8 | 22.6 | 39.8 | 28.7 | 3.7 |
| 15.1 | 12.1 | 23.0 | 20.7 | | |

TABLE 3
¹³C NMR Chemical Shifts from FIG. 1B for Form I.

| | Chemical |
|---|---|
| Peak | Shift (ppm) |
| 1 | 166.3 |
| 2 | 153.9 |
| 3 | 152.9 |
| 4 | 146.1 |
| 5 | 140.6 |
| 6 | 136.7 |
| 7 | 135.4 |
| 8 | 132.3 |
| 9 | 131.3 |
| 10 | 130.0 |
| 11 | 129.5 |
| 12 | 123.2 |
| 13 | 122.5 |
| 14 | 111.4 |
| 15 | 110.5 |
| 16 | 71.8 |
| 17 | 70.3 |
| 18 | 66.8 |
| 19 | 66.5 |
| 20 | 65.2 |
| 21 | 52.7 |
| 22 | 44.2 |
| 23 | 31.5 |
| 24 | 29.3 |
| 25 | 24.3 |
| 26 | 14.9 |
| 27 | 14.1 |
| 28 | 13.3 |

TABLE 4
Raman Peaks and Intensities for Form I (FIG. 1E).

| Position | Intensity | Position | Intensity | Position | Intensity |
|---|---|---|---|---|---|
| 220.1 | 544778.4 | 726.0 | 266585.7 | 1122.1 | 295339.8 |
| 239.8 | 733500.2 | 810.2 | 320890.5 | 1163.3 | 249939.9 |
| 272.9 | 486408.6 | 827.2 | 385122.2 | 1203.8 | 337894.6 |
| 327.1 | 453183.0 | 882.8 | 268777.5 | 1269.3 | 341976.7 |
| 443.5 | 344466.9 | 896.8 | 246982.3 | 1308.6 | 497437.5 |
| 532.3 | 304908.1 | 939.0 | 259511.5 | 1359.1 | 333532.8 |
| 556.4 | 368748.6 | 953.2 | 260280.5 | 1377.6 | 682867.3 |
| 597.0 | 304590.6 | 970.8 | 252256.6 | 1449.0 | 611815.3 |
| 626.5 | 288168.6 | 990.9 | 882145.3 | 1470.8 | 471266.7 |
| 644.2 | 332548.2 | 1022.4 | 274042.9 | 1572.5 | 523866.0 |
| 657.5 | 561207.8 | 1085.1 | 346739.8 | 1594.7 | 947833.4 |
| 681.3 | 373006.1 | 1107.3 | 284762.1 | | |

In one embodiment of the invention, Form I of Compound 1 is characterized by the XRPD pattern of FIG. 1A.

In another embodiment of the invention, Form I of Compound 1 has the XRPD characteristics shown in Table 2.

In another embodiment of the invention, Form I of Compound 1 is characterized by at least three XRPD peaks at 2Θ angles selected from 4.1°, 8.2°, 11.9°, 17.0°, 21.8°, 22.6° and 26.20.

In another embodiment of the invention, Form I of Compound 1 is characterized by at least five XRPD peaks at 2Θ angles selected from 4.1°, 8.2°, 11.9°, 17.0°, 21.8°, 22.6° and 26.2°.

In another embodiment of the invention, Form I of Compound 1 is characterized by XRPD peaks at 2Θ angles selected from 4.1°, 8.2°, 11.9°, 17.0°, 21.8°, 22.6° and 26.2°.

In one embodiment of the invention, Form I of Compound 1 is characterized by the ¹³C solid state NMR spectrum of FIG. 1B.

In another embodiment of the invention, Form I of Compound 1 has the ¹³C solid state NMR characteristics shown in Table 3.

In another embodiment of the invention, Form I of Compound 1 is characterized by ¹³C solid-state nuclear magnetic resonance peaks at chemical shifts selected from 166.3 ppm, 146.1 ppm, 65.2 ppm, 52.7 ppm and 44.2 ppm.

In another embodiment of the invention, Form I of Compound 1 is characterized by ¹³C solid-state nuclear magnetic resonance peaks at chemical shifts selected from 166.3 ppm, 152.9 ppm, 146.1 ppm, 140.6 ppm, 65.2 ppm, 52.7 ppm, 44.2 ppm, 31.5 ppm and 29.3 ppm.

In one embodiment of the invention, Form I of Compound 1 is characterized by thermal analysis profiles determined by DSC as shown in FIG. 1C. There is one endothermal event, the melting point of Compound I, which onsets at 200° C., and has a peak temperature of 202° C.

In another embodiment of the invention, Form I of Compound 1 is characterized by thermal analysis profiles determined by TGA as shown in FIG. 1D, which shows no significant loss on heating (>0.5%) up to 200° C., the melting point of Compound 1.

In one embodiment of the invention, Form I of Compound 1 is characterized by Raman as shown in FIG. 1E

In another embodiment of the invention, Form I of Compound 1 has the Raman characteristics shown in Table 4.

Samples of Form I were maintained for at least 9 months at 25°, and 60% and at least 6 months at 40° and 75% relative humidity. Under both storage conditions, the samples of Form I showed no increase in water content, no increase in impurity levels, no change in particle sized distribution, or changes in Raman spectra.

### Characteristics of Form III

The X-ray powder diffraction (XRPD) pattern of Form III of Compound 1 is shown in FIG. 2A; the ¹³C solid state NMR spectrum of Form III of Compound 1 is shown in FIG. 21B; and the thermal analysis profiles of Form Ill of Compound 1 determined by DSC and TGA measurement are shown in FIGS. 2C and 2D, and the Raman spectrum of Form Ill of Compound 1 is shown in FIG. 2E respectively.

Characteristic XRPD peaks, ¹³C solid-state nuclear magnetic resonance peaks and Raman peaks for Form Ill are provided in Table 5, Table 6 and Table 7, respectively.

TABLE 5
X-ray powder diffraction (XRPD) characteristics
from FIG. 2A for Form III.

| | Intensity I/I, | | Intensity I/I, | | Intensity I/I, |
|---|---|---|---|---|---|
| 2Θ, [°] | [%] | 2Θ, [°] | [%] | 2Θ, [°] | [%] |
| 3.9 | 15.9 | 16.6 | 32.6 | 23.2 | 15.5 |
| 7.7 | 100 | 17.3 | 13.6 | 23.8 | 14.4 |
| 11.5 | 31.3 | 18.3 | 15.2 | 24.4 | 5.9 |
| 12.5 | 66.9 | 19.8 | 9.5 | 25.2 | 6.0 |
| 14.3 | 5.6 | 20.4 | 12.4 | 26.7 | 12.1 |
| 14.6 | 4.4 | 21.4 | 19.8 | 27.2 | 4.5 |
| 15.4 | 9.8 | 22.9 | 28.9 | | |

TABLE 6
¹³C NMR Chemical Shifts from FIG. 2D for Form III.

| | Chemical |
|---|---|
| Peak | Shift (ppm) |
| 1 | 169.1 |
| 2 | 168.4 |
| 3 | 167.4 |
| 4 | 154.2 |
| 5 | 153.6 |
| 6 | 152.4 |
| 7 | 151.5 |
| 8 | 145.0 |
| 9 | 143.9 |
| 10 | 142.8 |
| 11 | 139.4 |
| 12 | 137.8 |
| 13 | 136.6 |
| 14 | 135.3 |
| 15 | 132.7 |
| 16 | 131.6 |
| 17 | 129.0 |
| 18 | 128.5 |
| 19 | 123.6 |
| 20 | 122.9 |
| 21 | 114.1 |
| 22 | 113.2 |
| 23 | 110.8 |
| 24 | 74.4 |
| 25 | 73.8 |
| 26 | 73.0 |
| 27 | 70.4 |
| 28 | 67.7 |
| 29 | 66.8 |
| 30 | 65.2 |
| 31 | 61.7 |
| 32 | 60.6 |
| 33 | 51.4 |
| 34 | 47.7 |
| 35 | 45.9 |
| 36 | 42.7 |
| 37 | 42.0 |
| 38 | 41.7 |
| 39 | 29.8 |
| 40 | 27.3 |
| 41 | 25.1 |
| 42 | 24.6 |
| 43 | 15.5 |
| 44 | 14.8 |
| 45 | 13.1 |

TABLE 7
Raman Peaks and Intensities for Form III (FIG. 2E).

| Position | Intensity | Position | Intensity | Position | Intensity |
|---|---|---|---|---|---|
| 198.1 | 672913.4 | 811.2 | 516796.4 | 1186.6 | 382813.6 |
| 242.1 | 741039.6 | 838.0 | 396499.5 | 1204.0 | 430624.5 |
| 328.5 | 575590.6 | 884.3 | 390866.0 | 1255.1 | 391750.9 |
| 442.2 | 494154.8 | 937.3 | 376747.6 | 1271.4 | 438806.9 |
| 462.7 | 488665.8 | 952.4 | 378641.3 | 1307.1 | 632964.1 |
| 531.5 | 469574.6 | 990.9 | 875840.3 | 1381.2 | 563637.6 |
| 554.5 | 481650.4 | 1013.8 | 388678.8 | 1392.0 | 576857.1 |
| 596.9 | 452717.9 | 1086.1 | 425919.1 | 1451.1 | 670645.8 |
| 614.9 | 457225.6 | 1108.0 | 359850.1 | 1472.0 | 568425.7 |
| 657.5 | 700281.5 | 1130.4 | 347371.1 | 1570.5 | 586304.0 |
| 732.0 | 414059.5 | 1159.2 | 349294.7 | 1594.4 | 844826.9 |

In one embodiment of the invention, Form Ill of Compound 1 is characterized by the XRPD pattern of FIG. 2A.

In another embodiment of the invention, Form III of Compound 1 has the XRPD characteristics shown in Table 4.

In another embodiment of the invention, Form III of Compound 1 is characterized by at least three XRPD peaks at 2Θ angles selected from 7.7°, 11.5°, 12.5°, 16.6° and 22.9°.

In another embodiment of the invention, Form III of Compound 1 is characterized by XRPD peaks at 2Θ angles selected from 7.7°, 11.5°, 12.5°, 16.6° and 22.9°.

In one embodiment of the invention, Form III of Compound 1 is characterized by the ¹³C solid state NMR spectrum of FIG. 2B.

In another embodiment of the invention, Form III of Compound 1 has the ¹³C solid state NMR characteristics shown in Table 5.

In another embodiment of the invention, Form III of Compound 1 is characterized by ¹³C solid-state nuclear magnetic resonance peaks at chemical shifts selected from 168.4 ppm, 167.4 ppm, 151.5 ppm, 60.6 ppm, 51.4 ppm, 47.7 ppm and 42.7 ppm.

In another embodiment of the invention, Form III of Compound 1 is characterized by ¹³C solid-state nuclear magnetic resonance peaks at chemical shifts selected from 168.4 ppm, 167.4 ppm, 151.5 ppm, 143.9 ppm, 142.8 ppm, 137.8 ppm, 113.2 ppm, 110.8 ppm, 73.0 ppm, 65.2 ppm, 61.7 ppm, 60.6 ppm, 51.4 ppm, 47.7 ppm, 42.7 ppm, 42.0 ppm and 41.7 ppm.

In one embodiment of the invention, Form III of Compound 1 is characterized by thermal analysis profiles determined by DSC as shown in FIG. 2C. There are two features in the DSC: one endotherm with an onset at 83° C., and a peak temperature of 98° C. Followed by a second endotherm that onsets at 195° C., with a peak temperature of 199° C.

In another embodiment of the invention, Form III of Compound 1 is characterized by thermal analysis profiles determined by TGA as shown in FIG. 2D, with a 3.6% loss of heating from 25° C. to 100° C. (prior to the melt of Compound I, Form III).

In one embodiment of the invention, Form III of Compound 1 is characterized by Raman as shown in FIG. 2E.

In another embodiment of the invention, Form III of Compound 1 has the Raman characteristics shown in Table 7.

### Characteristics of Form IV

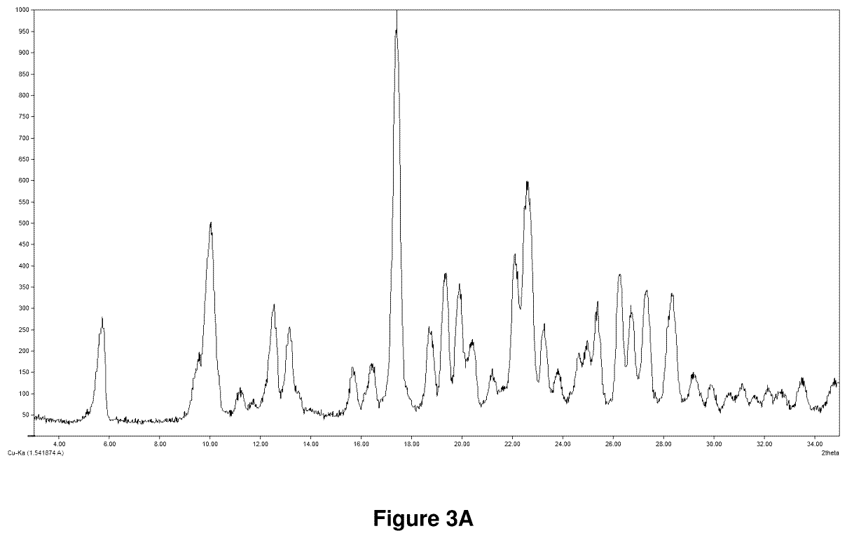

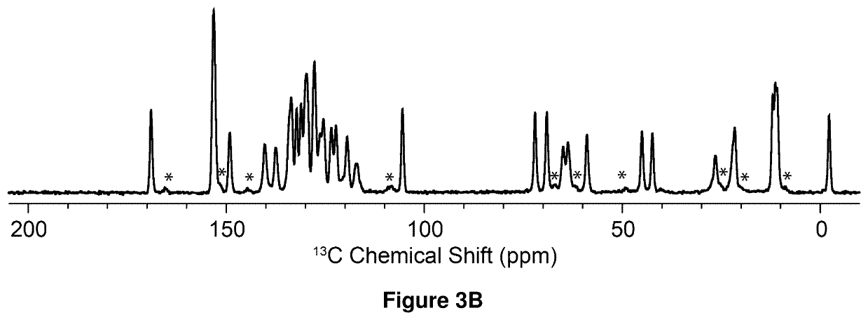

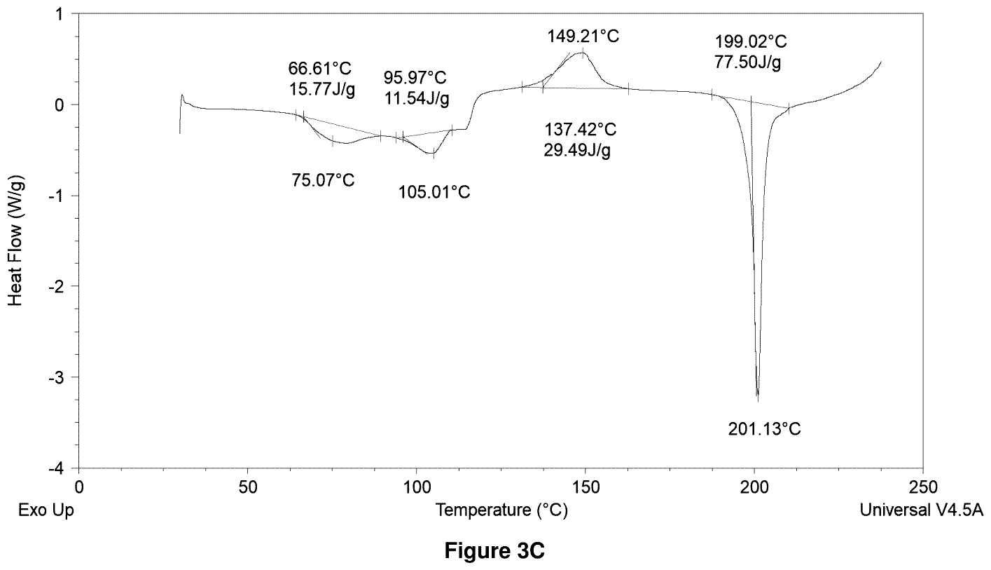

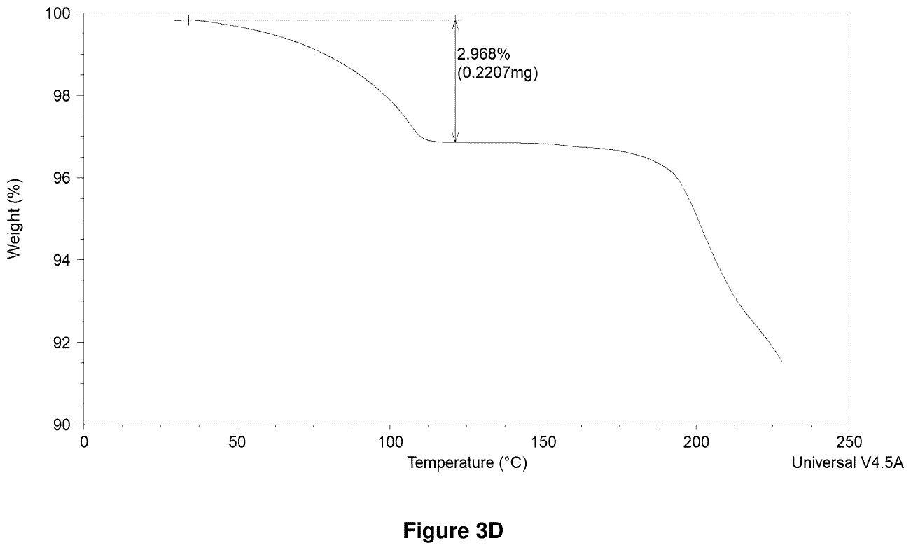

The X-ray powder diffraction (XRPD) pattern of Form IV of Compound 1 is shown in FIG. 3A; the ¹³C solid state NMR spectrum of Form IV of Compound 1 is shown in FIG. 31B; and the thermal analysis profiles of Form IV of Compound 1 determined by DSC and TGA measurement are shown in FIGS. 30 and 3D, respectively.

Characteristic XRPD peaks and ¹³C solid-state nuclear magnetic resonance peaks for Form IV are provided in Table 8 and Table 9, respectively.

TABLE 8
X-ray powder diffraction (XRPD) characteristics
from FIG. 3A for Form IV.

| | Intensity I/I, | | Intensity I/I, | | Intensity I/I, |
|---|---|---|---|---|---|
| 2Θ, [°] | [%] | 2Θ, [°] | [%] | 2Θ, [°] | [%] |
| 5.7 | 25.6 | 19.9 | 29.2 | 27.3 | 28.0 |
| 10.0 | 47.7 | 20.4 | 15.9 | 28.3 | 27.2 |
| 11.2 | 5.0 | 21.2 | 7.8 | 29.2 | 7.6 |
| 11.7 | 2.4 | 22.1 | 33.1 | 29.9 | 5.0 |
| 12.5 | 26.0 | 22.6 | 56.8 | 30.6 | 3.3 |
| 13.1 | 20.7 | 23.4 | 17.5 | 31.1 | 5.1 |
| 15.7 | 10.9 | 23.8 | 8.4 | 32.1 | 4.5 |
| 16.4 | 11.4 | 25.0 | 14.8 | 32.6 | 3.5 |
| 17.4 | 100 | 25.4 | 14.5 | 33.5 | 6.4 |
| 18.7 | 20.2 | 26.3 | 31.7 | | |
| 19.3 | 34.4 | 26.7 | 20.6 | | |

TABLE 9
¹³C NMR Chemical Shifts from FIG. 3B for Form IV.

| | Chemical |
|---|---|
| Peak | Shift (ppm) |
| 1 | 169.8 |
| 2 | 154.4 |
| 3 | 150.4 |
| 4 | 141.8 |
| 5 | 139.1 |
| 6 | 135.8 |
| 7 | 135.2 |
| 8 | 133.9 |
| 9 | 132.8 |
| 10 | 131.7 |
| 11 | 131.4 |
| 12 | 129.5 |
| 13 | 128.1 |
| 14 | 127.3 |
| 15 | 125.4 |
| 16 | 124.2 |
| 17 | 121.5 |
| 18 | 119.2 |
| 19 | 119.0 |
| 20 | 107.8 |
| 21 | 75.1 |
| 22 | 72.2 |
| 23 | 68.2 |
| 24 | 67.0 |
| 25 | 62.3 |
| 26 | 48.8 |
| 27 | 46.2 |
| 28 | 30.6 |
| 29 | 26.3 |
| 30 | 25.9 |
| 31 | 16.5 |
| 32 | 15.9 |
| 33 | 15.4 |
| 34 | 2.6 |

In one embodiment of the invention, Form IV of Compound 1 is characterized by the XRPD pattern of FIG. 3A.

In another embodiment of the invention, Form IV of Compound 1 has the XRPD characteristics shown in Table 8.

In another embodiment of the invention, Form IV of Compound 1 is characterized by at least three XRPD peaks at 2Θ angles selected from 5.7°, 10.0°, 17.4°, 22.6°, and 28.3°.

In another embodiment of the invention, Form IV of Compound 1 is characterized by XRPD peaks at 2Θ angles selected from 5.7°, 10.0°, 17.4°, 22.6°, and 28.3°.

In another embodiment of the invention, Form IV of Compound 1 is characterized by XRPD peaks at 2Θ angles selected from 5.7°, 10.0°, 12.5°, 13.1, 17.4°, 22.6°, 26.3°, 27.3°, and 28.30.

In one embodiment of the invention, Form IV of Compound 1 is characterized by the ¹³C solid state NMR spectrum of FIG. 3B.

In another embodiment of the invention, Form IV of Compound 1 has the ¹³C solid state NMR characteristics shown in Table 9.

In another embodiment of the invention, Form IV of Compound 1 is characterized by ¹³C solid-state nuclear magnetic resonance peaks at chemical shifts selected from 169.8 ppm, 107.8 ppm, 30.6 ppm and 2.6 ppm.

In another embodiment of the invention, Form IV of Compound 1 is characterized by ¹³C solid-state nuclear magnetic resonance peaks at chemical shifts selected from 169.8 ppm, 139.1 ppm, 107.8 ppm, 75.1 ppm, 30.6 ppm and 2.6 ppm.

In one embodiment of the invention, Form IV of Compound 1 is characterized by thermal analysis profiles determined by DSC as shown in FIG. 3C. There are two consecutive endotherms with peak temperatures of 75° C. (onset: 67° C.), and 105° C. (onset 96° C.). At 137° C. there is an exotherm which has a peak temperature of 149° C., followed by a final endotherm onsetting at 199° C., with a peak temperature of 201° C.

In another embodiment of the invention, Form IV of Compound 1 is characterized by thermal analysis profiles determined by TGA as shown in FIG. 3D. The total loss on heating from 25° C. to 125° C. is 2.9%

### Characteristics of Form V

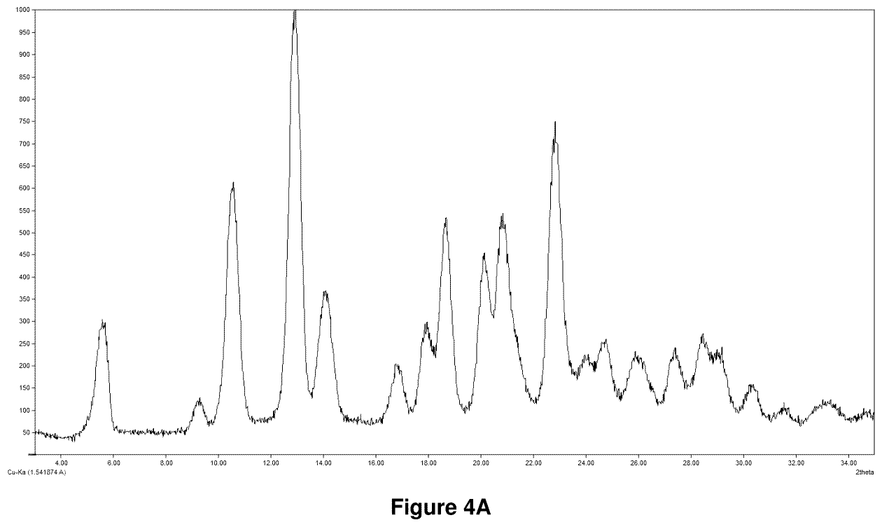

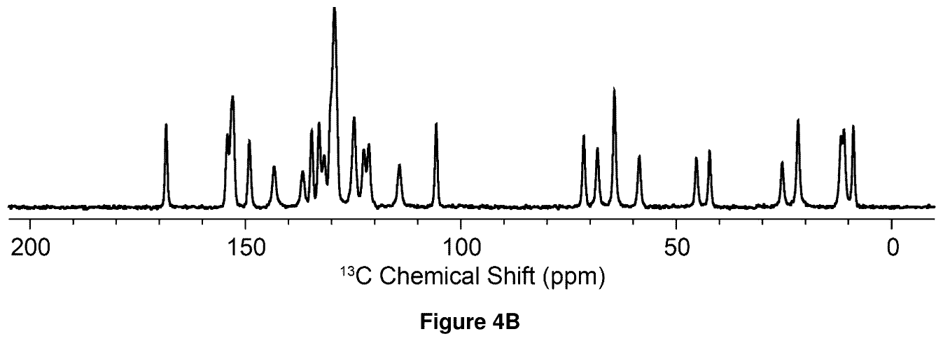

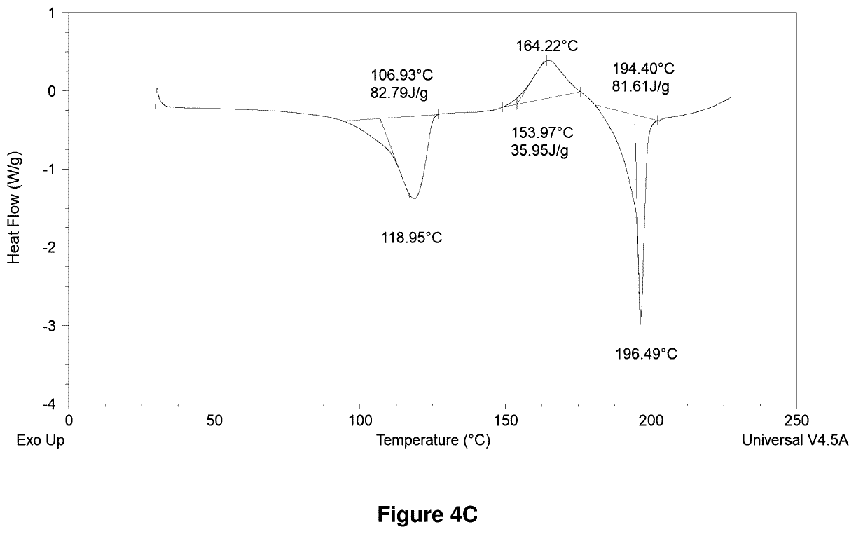

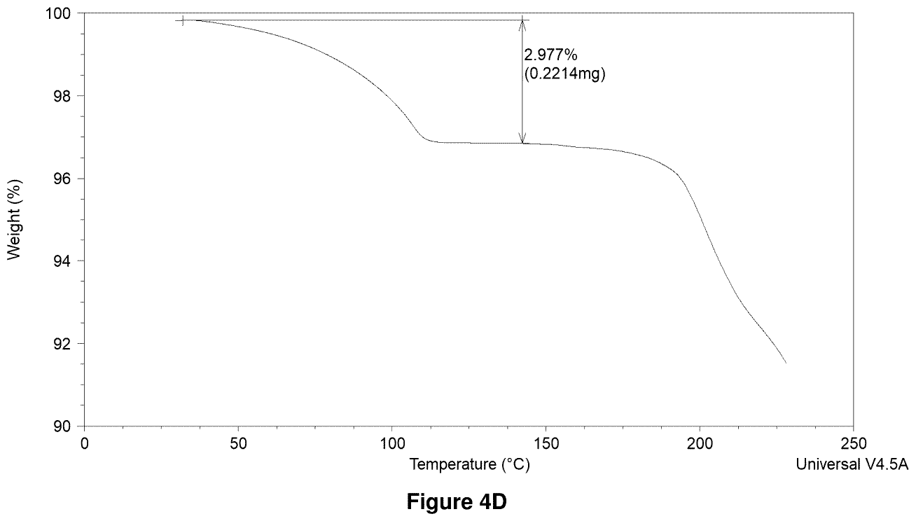

The X-ray powder diffraction (XRPD) pattern of Form V of Compound 1 is shown in FIG. 4A; the ¹³C solid state NMR spectrum of Form V of Compound 1 is shown in FIG. 4B; and the thermal analysis profiles of Form V of Compound 1 determined by DSC and TGA measurement are shown in FIGS. 4C and 4D, respectively.

Characteristic XRPD peaks and ¹³C solid-state nuclear magnetic resonance peaks for Form V are provided in Table 10 and Table 11, respectively.

TABLE 10
X-ray powder diffraction (XRPD) characteristics
from FIG. 4A for Form V.

| | Intensity I/I, | | Intensity I/I, | | Intensity I/I, |
|---|---|---|---|---|---|
| 2Θ, [°] | [%] | 2Θ, [°] | [%] | 2Θ, [°] | [%] |
| 5.6 | 27.2 | 17.9 | 19.6 | 25.9 | 13.7 |
| 9.3 | 6.5 | 18.7 | 45.5 | 27.4 | 13.8 |
| 10.5 | 58.3 | 20.1 | 30.9 | 28.5 | 14.5 |
| 12.9 | 100 | 20.8 | 43.7 | 29.0 | 11.3 |
| 14.1 | 31.0 | 22.8 | 67.1 | 30.3 | 3.4 |
| 16.8 | 12.9 | 24.0 | 10.5 | | |

TABLE 11
¹³C NMR Chemical Shifts from FIG. 4B for Form V.

| | Chemical |
|---|---|
| Peak | Shift (ppm) |
| 1 | 169.3 |
| 2 | 155.4 |
| 3 | 154.5 |
| 4 | 154.1 |
| 5 | 150.4 |
| 6 | 144.8 |
| 7 | 138.3 |
| 8 | 136.3 |
| 9 | 134.6 |
| 10 | 133.4 |
| 11 | 131.9 |
| 12 | 131.1 |
| 13 | 126.6 |
| 14 | 124.4 |
| 15 | 123.3 |
| 16 | 116.4 |
| 17 | 108.0 |
| 18 | 74.6 |
| 19 | 71.5 |
| 20 | 67.6 |
| 21 | 62.0 |
| 22 | 49.0 |
| 23 | 46.0 |
| 24 | 29.6 |
| 25 | 26.0 |
| 26 | 16.2 |
| 27 | 15.6 |
| 28 | 13.5 |

In one embodiment of the invention, Form V of Compound 1 is characterized by the XRPD pattern of FIG. 4A.

In another embodiment of the invention, Form V of Compound 1 has the XRPD characteristics shown in Table 10.

In another embodiment of the invention, Form V of Compound 1 is characterized by at least three XRPD peaks at 2Θ angles selected from 5.6°, 10.5°, 12.90 and 22.8°.

In another embodiment of the invention, Form V of Compound 1 is characterized by XRPD peaks at 2Θ angles selected from 5.6°, 10.5°, 12.9°, 17.9°, 18.7°, 20.1°, 20.8° and 22.8°.

In one embodiment of the invention, Form V of Compound 1 is characterized by the ¹³C solid state NMR spectrum of FIG. 4B.

In another embodiment of the invention, Form V of Compound 1 has the ¹³C solid state NMR characteristics shown in Table 11.

In another embodiment of the invention, Form V of Compound 1 is characterized by ¹³C solid-state nuclear magnetic resonance peaks at chemical shifts selected from 155.4 ppm, 116.4 ppm, 74.6 ppm, 71.5 ppm and 29.6 ppm.

In another embodiment of the invention, Form V of Compound 1 is characterized by ¹³C solid-state nuclear magnetic resonance peaks at chemical shifts selected from 169.3, 155.4 ppm, 150.4 ppm, 116.4 ppm, 108.0 ppm, 74.6 ppm, 71.5 ppm, 67.7 ppm, 29.6 ppm and 26.0 ppm.

In one embodiment of the invention, Form V of Compound 1 is characterized by thermal analysis profiles determined by DSC as shown in FIG. 4C. There are multiple thermal events: first an endotherm with onset at 107° C. and peak temperature of 119° C., this is followed by an exotherm with onset at 154° C. (164° C. peak temperature), and finally an endotherm with a peak temperature of 196° C., which onsets at 194° C.

In another embodiment of the invention, Form V of Compound 1 is characterized by thermal analysis profiles determined by TGA as shown in FIG. 4D. The total loss on heating from 25° C. to 140° C. is 3.0%.

### Methods of Preparing the Compounds of the Invention

Specific conditions for the preparation of the solid forms of Compound 1 of the invention are described in the Examples. In general, the compounds of the invention may be obtained by dissolving Compound 1 in a suitable solvent (“the dissolution step”), preferably at a temperature above room temperature, more preferably from about 45° C. to about 80° C. The heated solution can then be cooled (“the cooling step”) to provide a solid/liquid comprising the compounds of the invention. In some embodiments, the heated solutions may be filtered prior to cooling. In other embodiments, the heated solutions may be concentrated prior to or during the cooling step (“the concentration step”). In still other embodiments, the heated solutions may be treated with a cosolvent (“the cosolvent treatment step”). In some embodiments, the cosolvent (when used) can be added during the cooling step. In still other embodiments, the cooling step comprises a step-wise cooling ramp. In still other embodiments, a seed crystal or seed slurry (“the seeding step”) is added prior to or during the cooling step. It is understood that any combinations of the above-may be used to obtain the compounds of the invention and mixtures thereof. Once cooled, the resulting solids can be collected, washed with a suitable solvent, and dried to provide the compounds of the invention, or mixtures thereof.

In one embodiment, Form I can be obtained using a reactive crystallization with pH swing. Compound 1 is dissolved in an aqueous basic solution (NaOH or ammonia). The pH is adjusted with acid (HCl or citric acid) and seeded with Form I. The pH is slowly neutralized with acid as the solid phase of the slurry grows. The solids are collected, washed with suitable solvent, and dried.

### Methods of Therapeutic Use

The compounds disclosed herein effectively activate soluble guanylate cyclase. The activation or potentiation of soluble guanylate cyclase is an attractive means for preventing and treating certain diseases and disorders. Nonlimiting examples of such diseases or disorders include those described in WO 2014/039434 and WO 2020/011804.

In another embodiment, the invention relates to methods of treating diseases and disorders including:
Cardiovascular and related diseases including hypertension, atherosclerosis, peripheral artery disease, restenosis, stroke, heart failure, coronary vasospasm, cerebral vasospasm, ischemia/reperfusion injury, thromboembolic pulmonary hypertension, pulmonary arterial hypertension, stable and unstable angina and thromboembolic disorders;
Inflammatory diseases including psoriasis, multiple sclerosis, arthritis, asthma, and chronic obstructive pulmonary disease; hepatic fibrotic disorders including but not limited to cirrhosis of any etiology or fibrosis of specific areas of the liver such as periportal fibrosis which may be caused by immunologic injury, hemodynamic effects and/or other causes;
Renal fibrotic disorders including but not limited to glomerulosclerosis, focal glomerulosclerosis, mesangial fibrosis, interstitial fibrosis due to immunologic injury, hemodynamic effects, diabetes (types I and 2), diabetic nephropathy, IgA nephropathy, lupus nephropathy, membranous nephropathy, hypertension, hemolytic uremic syndrome, multiple glomerulonephritides, interstitial nephritis, tubulointerstitial nephritis again of immunologic and non-immunologic causes;
Pulmonary fibrotic disorders, both diffuse and localized, due to immunologic and non-immunologic causes, including but not limited to idiopathic pulmonary fibrosis, pulmonary fibrosis due to exposure to toxins, chemicals, drugs, and cystic fibrosis;
Cardiac fibrotic disorders due to immunologic and non-immunologic causes including ischemic heart disease (coronary artery disease) and transient and/or sustained decreased blood flow in one or more coronary vessels including possibly related to interventions on coronary arteries or veins, associated with cardiac surgery and/or the use of cardiopulmonary bypass procedures and myocarditis due to viral and non-viral causes, as well as immunologically related myocardial injury potentially due to cross-reactivity to other antigens to which the human body is exposed;
Other diseases mediated at least partially by diminished or decreased soluble guanylate cyclase activity, such as renal disease, diabetes, urologic disorders including overactive bladder, benign prostatic hyperplasia, and erectile dysfunction, and neurological disorders including Alzheimer's disease, Parkinson's disease and neuropathic pain.

In another embodiment, the invention relates to the use the compounds of the invention to treat, alleviate, or slow the progression of chronic kidney disease (CKD), including fast progressors of CKD); non-alcoholic steatohepatitis (NASH); all-cause cirrhosis; clinically significant portal hypertension (CSPH), and systemic scleroderma (sclerosis).

According to one embodiment, the invention relates to a method for treating, preventing, slowing the progression of non-alcoholic steatohepatitis (NASH), in a patient in need thereof characterized in that a pharmaceutical composition or pharmaceutical dosage form as defined hereinbefore and hereinafter is administered to the patient.

In one embodiment, the compounds of the invention may be used for treating NASH with fibrosis, for example, F1 to F4.

In another embodiment, the compounds of the invention may be used for treating cirrhosis with and without clinically significant portal hypertension.

In another embodiment, the invention relates to treatment of patients with compensated NASH cirrhosis with clinically significant portal hypertension (CSPH). Portal venous pressure is the blood pressure in the hepatic portal vein, and is normally between 5-10 mmHg.

Raised portal venous pressure is termed portal hypertension, and has numerous sequelae such as ascites and hepatic encephalopathy. In one embodiment of the invention, CSPH is defined as a hepatic venous pressure gradient (HVPG)≥10 mm/Hg. Accordingly, another embodiment of the invention relates to treatment of patients with compensated NASH cirrhosis with a venous pressure gradient (HVPG)≥10 mm/Hg.

In another embodiment, the invention relates to treatment of portal hypertension in cirrhotic patients, where the cirrhosis is due to any etiology (all-cause cirrhosis). Etiologies include, but are not limited to, NASH, alcoholic liver disease (ALD), hepatitis C, hepatitis B, chronic primary biliary liver diseases (Primary Sclerosing Cholangitis, Primary Biliary Cirrhosis).

According to another aspect the present invention relates to a method for treating non-alcoholic steatohepatitis (NASH, NAS 4), in particular of NASH with liver fibrosis, for example of NASH with liver fibrosis stages 2 and 3, in a patient in need thereof characterized in that a pharmaceutical composition comprising a compound of formula (I) or a pharmaceutically acceptable salt thereof as an active pharmaceutical ingredient (API), preferably a pharmaceutical composition according to this invention, is administered to the patient.

In one embodiment, the invention relates to the use of a compound of the invention for the preparation of a medicament for treating, preventing, or slowing the progression of non-alcoholic steatohepatitis (NASH) or of one or more of the other conditions or diseases selected from the group of conditions or diseases as outlined above in this section entitled “Methods of Therapeutic Use”.

In one embodiment, the invention relates to a compound of the invention for use in treating, preventing, or slowing the progression of non-alcoholic steatohepatitis (NASH) or of one or more of the other conditions or diseases selected from the group of conditions or diseases as outlined above in this section entitled “Methods of Therapeutic Use”.

Another aspect of the invention concerns the compounds of the invention for use in the manufacture of a medicament for the treatment of a condition or a disease selected from the group of conditions or diseases as outlined above in this section entitled “Methods of Therapeutic Use”.

The effect of an administration of said pharmaceutical composition to a patient with NASH and/or liver fibrosis may be observed by a change, in particular reduction of relevant biomarkers of liver inflammation and/or liver function, such as for example ALT (alanine aminotransferase), AST (aspartate aminotransferase), AP (alkaline phosphatase), gamma-GT (gamma-glutamil transferase), CK-18 (cytokeratin 18) fragments or HVPG (hepatic vein pressure gradient).

Furthermore the effect of an administration of said pharmaceutical composition to a patient with NASH and/or liver fibrosis may be observed by an improvement of for example the degree or stage of steatosis, fibrosis, liver stiffness or health-related quality of life.

For therapeutic use, the compounds of the invention may be administered via a pharmaceutical composition in any conventional pharmaceutical dosage form in any conventional manner. Conventional dosage forms typically include a pharmaceutically acceptable carrier suitable to the particular dosage form selected. Routes of administration include, but are not limited to, intravenously, intramuscularly, subcutaneously, intrasynovially, by infusion, sublingually, transdermally, orally, topically or by inhalation. The preferred modes of administration are oral and intravenous.

Preferred doses of the compound of the invention for oral administration are 0.1 to 100 mg; or 1 to 25 mg; or 1 to 10 mg; or 1 to 5 mg. In another embodiment, the preferred doses of the compound of the invention for oral administration for oral administration are selected from 1 mg, 1.5 mg, 2 mg, 2.5 mg, 3 mg, 3.5 mg, 4, mg, 4.5 mg, 5 mg, 5.5 mg, 6 mg, 6.5 mg, 7 mg, 7.5 mg, 8 mg, 8.5 mg, 9 mg, 9.5 mg, and 10 mg.

In one embodiment, the compound of the invention may be administered once per day, twice per day, or three or more times per day. In another embodiment, the compound of the invention may be administered once per week, twice per week, or three or more times per week.

The compounds of this invention may be administered alone or in combination with adjuvants that enhance stability of the inhibitors, facilitate administration of pharmaceutical compositions containing them in certain embodiments, provide increased dissolution or dispersion, increase inhibitory activity, provide adjunct therapy, and the like, including other active ingredients. In one embodiment, for example, multiple compounds of the present invention can be administered. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds of the present invention and the conventional therapeutics or other adjuvants may be administered separately (either serially or in parallel). Separate dosing allows for greater flexibility in the dosing regimen.

As mentioned above, dosage forms of the compounds of this invention may include pharmaceutically acceptable carriers and adjuvants known to those of ordinary skill in the art and suitable to the dosage form. These carriers and adjuvants include, for example, ion exchangers, alumina, aluminum stearate, lecithin, serum proteins, buffer substances, water, salts or electrolytes and cellulose-based substances. Preferred dosage forms include tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements for the compounds of the present invention may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

In one embodiment, for example, multiple compounds of the present invention can be administered. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds of the present invention and the conventional therapeutics or other adjuvants may be administered separately (either serially or in parallel). Separate dosing allows for greater flexibility in the dosing regimen.

### EXAMPLES

The amorphous form of Compound 1 (“amorphous Compound 1”) is prepared as described in Example 114 of WO 2014/039434. Solvents are removed from the resulting eluent under reduced pressure to provide amorphous Compound 1 as a solid. A typical XRPD pattern obtained for amorphous Compound 1 is shown in FIG. 5.

### Example 1a

### Preparation of Form I of Compound 1

The amorphous form of Compound 1 (1.2 g) is treated with MeOH (12 g) and heated with stirring to 60° C. The resulting slurry is treated with water (1 mL) and THF (8 mL). The mixture is stirred for an additional 1 hour at 60° C., cooled to 20° C. over 4 hours, and stirred overnight at 20° C. The solids are collected by filtration and dried to provide Form I of Compound 1 (1.03 g).

### Example 1b

### Preparation of Form I of Compound 1

The amorphous form of Compound 1 is treated with concentrated aqueous hydrochloric acid in THF and water (THF/H₂O 97/3) to provide the HCl salt of Compound 1. The HCl salt of Compound 1 (4.45 kg) is treated with 2 equivalents of NaOH in water (1.15 kg of 50% NaOH in water) to provide the sodium salt of Compound 1 salt as a clear solution. The solution is polish filtered and the pH of the filtrate is adjusted with dilute aqueous HCl solution provide a pH of the solution of 8.6 to 9.0. The solution is then seeded with Form 1 of Compound 1 (0.004 kg) obtained as described above to initiate crystallization. The seeded mixture is treated with additional amounts of dilute HCl until the pH of the mixture reaches approximately 7.2-7.9. The reaction mixture is cooled to 20° C., aged for several hours and centrifuge filtered. The solids are washed with water (110 kg) followed by acetone (6.8 kg). The solid is then dried under reduced pressure with a nitrogen stream at about 40° C. to provide Form I (4.08 kg).

### Example 1c

### Preparation of Form I of Compound 1

The amorphous form of Compound 1 (7 g) is suspended in EtOH (56 mL) and water (31 ml). To this, 7 g of ammonia solution (25% w/w) is added over 5 min, to form a clear, pale yellow solution. The solution is heated with stirring to 50° C., and held for 15 min. Citric acid (11 g of 50% w/w solution) is added) over 45 min, until the pH reaches a range of 8.0-7.2. The solution is seeded with Form 1 (7 mg, prepared as described above), and the mixture is held for 15 min. An additional 2.65 g of citric acid solution (50% w/w) is then added over 120 min, until a pH of approx. 6.0 is achieved. The resulting slurry is cooled to 25° C. over 60 minutes. The solids are collected by filtration, washed with 50 mL of H₂O and dried to provide Form I of Compound 1 (6.723 g).

### Example 2a

### Preparation of Form III of Compound 1

The amorphous of Compound 1 (10 mg) is added to a pan for Dynamic Vapor Sorption analysis. The solid is exposed stepwise from 40 to 95 to 0 to 95 to 0% RH at 25° C. Form III is isolated after completing the stepwise exposures.

### Example 2b

### Preparation of Form III of Compound 1

The amorphous form of Compound 1 (100 mg) is treated with water (1.0 mL). The mixture is agitated with a magnetic stir bar at room temperature and solids collected to provide Form III.

### Example 3a

### Preparation of Form IV of Compound 1

Form I of Compound 1 (1 g) is dissolved in 5 mL of 70% water in acetonitrile (ACN) to form a saturated solution. The solution is left to stir for 72 hours with a magnetic stir bar at room temperature. The resulting crystals are collected as Form IV. Stoichiometric ratios of Form IV (Compound 1: ACN:H2O, 1:1:1) are determined by Single Crystal X-ray diffraction.

### Example 3b

### Preparation of Form IV of Compound 1

Form I of Compound 1 (1 g) is treated with in 5 mL of 70% water in acetonitrile (ACN). The mixtures is agitated for 48 hours with a magnetic stir bar at room temperature. The resulting crystals are collected as Form IV.

### Example 4a

### Preparation of Form V of Compound 1

Form IV crystals (prepared as described above) are left on the benchtop to air dry at room temperature for 24 hours to provide Form V. Stoichiometric ratios of Form V (Compound 1: H2O, 1:1) were determined by Single Crystal X-ray.

### Example 4b

### Preparation of Form V of Compound 1

Form I of Compound 1 (2 g) is treated with 10 mL of 70% water in acetonitrile (ACN) and seeded with Form IV from Example 3b. The mixture is agitated for 24 hours with a magnetic stir bar at room temperature. The solids are then isolated by filtration and dried overnight at 60° C. under reduced pressure to provide Form V.

What is claimed is:

1. A solid crystalline form of Compound 1: 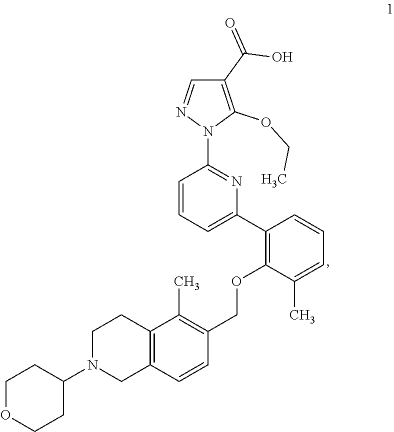 [SMILES: CCOc1c(C(=O)O)cnn1-c1cccc(-c2cccc(C)c2OCc2ccc3c(c2C)CCN(C2CCOCC2)C3)n1]
wherein the solid crystalline form of Compound 1 is selected from the group consisting of:
i) Form I characterized by:
at least three XRPD peaks at 2Θ angles selected from 4.1°, 8.2°, 11.9°, 17.0°, 21.8°, 22.6° and 26.2°; or
¹³C solid-state nuclear magnetic resonance peaks at chemical shifts selected from 166.3 ppm, 146.1 ppm, 65.2 ppm, 52.7 ppm and 44.2 ppm;
ii) Form III characterized by:
at least three XRPD peaks at 2° angles selected from 7.7°, 11.5°, 12.5°, 16.6° and 22.9°; or
¹³C solid-state nuclear magnetic resonance peaks at chemical shifts selected from 168.4 ppm, 167.4 ppm, 151.5 ppm, 60.6 ppm, 51.4 ppm, 47.7 ppm and 42.7 ppm;
iii) Form IV characterized by:
at least three XRPD peaks at 2° angles selected from 5.7°, 10.0°, 17.4°, 22.6°, and 28.3°; or
¹³C solid-state nuclear magnetic resonance peaks at chemical shifts selected from 169.8 ppm, 107.8 ppm, 30.6 ppm and 2.6 ppm; and
iv) Form V characterized by:
at least three XRPD peaks at 2° angles selected from 5.6°, 10.5°, 12.9° and 22.8°;
or
¹³C solid-state nuclear magnetic resonance peaks at chemical shifts selected from 155.4 ppm, 116.4 ppm, 74.6 ppm, 71.5 ppm and 29.6 ppm

2. Form I of the solid crystalline form of Compound 1 of claim 1, further characterized by:
XRPD peaks at 2° angles selected from 4.1°, 8.2°, 11.9°, 17.0°, 21.8°, 22.6° and 26.2°; or
¹³C solid-state nuclear magnetic resonance peaks at chemical shifts selected from 166.3 ppm, 152.9 ppm, 146.1 ppm, 140.6 ppm, 65.2 ppm, 52.7 ppm, 44.2 ppm, 31.5 ppm and 29.3 ppm.

3. Form III of the solid crystalline form of Compound 1 of claim 1, further characterized by:
XRPD peaks at 2Θ angles selected from 7.7°, 11.5°, 12.5°, 16.6° and 22.9°; or
¹³C solid-state nuclear magnetic resonance peaks at chemical shifts selected from 168.4 ppm, 167.4 ppm, 151.5 ppm, 143.9 ppm, 142.8 ppm, 137.8 ppm, 113.2 ppm, 110.8 ppm, 73.0 ppm, 65.2 ppm, 61.7 ppm, 60.6 ppm, 51.4 ppm, 47.7 ppm, 42.7 ppm, 42.0 ppm and 41.7 ppm.

4. Form IV of the solid crystalline form of Compound 1 of claim 1, further characterized by:
XRPD peaks at 2Θ angles selected from 5.7°, 10.0°, 12.5°, 13.1, 17.4°, 22.6°, 26.3°, 27.3°, and 28.3°; or
¹³C solid-state nuclear magnetic resonance peaks at chemical shifts selected from 169.8 ppm, 139.1 ppm, 107.8 ppm, 75.1 ppm, 30.6 ppm and 2.6 ppm.

5. Form V of the solid crystalline form of Compound 1 of claim 1, further characterized by:
XRPD peaks at 2° angles selected from 5.6°, 10.5°, 12.9°, 17.9°, 18.7°, 20.1°, 20.8° and 22.8°.

6. A pharmaceutical composition comprising any of Form I, Form III or Form V according to claim 1, optionally together with one or more inert carriers and/or diluents.

7. A pharmaceutical composition comprising Form I according to claim 2, optionally together with one or more inert carriers and/or diluents.

8. A pharmaceutical composition comprising Form III according to claim 4, optionally together with one or more inert carriers and/or diluents.

9. A pharmaceutical composition comprising Form V according to claim 5, optionally together with one or more inert carriers and/or diluents.

10. A method for treating and/or preventing a disease or disorder that is responsive to treatment with an activator of sGC comprising administering a pharmaceutically effective amount of Form I, Form III or Form V according to claim 1 to a patient in need thereof.

11. The method according to claim 10, wherein the diseases or disorders that are responsive to treatment with an activator of sGC is selected from the group consisting of chronic kidney disease, diabetic kidney disease, nonalcoholic steatohepatitis (NASH), liver cirrhosis, portal hypertension, and systemic sclerosis (scleroderma).

12. A method of producing Form I of Compound 1 of claim 1, comprising:
(i) adding aqueous base to a suspension of Compound 1, ethanol and water to provide a solution,
(ii) heating the solution of step (I) to 50° C.,
(iii) treating the solution of step (ii) with acid until the pH reaches a range of 8.0-7.2,
(iv) adding a seed crystal of Form I of Compound 1 to the solution of step (iii) to provide a seeded mixture,
(v) treating the seeded mixture of step ((iv) with acid until the pH reaches 6.0,
(vi) cooling the mixture of step (v), and
(vii) isolating the solids from the mixture of step (vi) to provide Form I of Compound 1.
